(19) United States
(12) Patent Application Publication
(10) Pub. No.: US 2010/0298771 A1
(43) Pub. Date: Nov. 25, 2010

(54) SYSTEM FOR CHEMOHYPERTHERMIA TREATMENT

(75) Inventor: Aik Ping Theodore Tan, Singapore (SG)

Correspondence Address: HESLIN ROTHENBERG FARLEY & MESITI PC, 5 COLUMBIA CIRCLE, ALBANY, NY, 12203, US

(73) Assignee: DYAMED BIOTECH PTE LTD, (SG)

(21) Appl. No.: 12/446,347

(22) PCT Filed: Oct. 12, 2007
(86) PCT No.: PCT/SG07/00347

§ 371 Date: May 21, 2009

(30) Foreign Application Priority Data
Oct. 19, 2006 (US) 11/551235

(51) Int. Cl. A61F 7/12

(52) U.S. Cl. 604/113; 607/105

(57) ABSTRACT

A chemohyperthermia treatment system includes a reservoir for storing fluid, a heating/cooling system coupled to the reservoir so that the fluid can be transferred from the reservoir to the heating system, wherein the heating/cooling system comprises a heating/cooling exchange module having a channel within which the fluid can flow, and a plurality of peltier modules/heating strips coupled to the heating/cooling module, wherein the plurality of peltier modules/heating strips heat up the fluid flowing through the channel, and wherein in the cooling mode, the plurality of peltier modules cool the fluid flowing through the channel. A pumping means is coupled to the heating/cooling system, wherein the pumping means pump the perfusion fluid from the reservoir to the heating/cooling system, thereby allowing the heating/cooling system to change the temperature of the fluid. A least one inflow catheter is coupled to the pumping means, wherein the at least one inflow catheter delivers the heated/cooled fluid to an object. At least one outflow catheter coupled to the reservoir, wherein the at least one outflow catheter drains the fluid from the object to the reservoir.

### FIELD OF THE INVENTION

The present invention generally relates to hyperthermia treatment, and more particularly to an intracavitary chemohyperthermia system that provides stable heating of perfusion fluid.

### BACKGROUND OF THE INVENTION

Hyperthermia treatment generally refers to a process for treating certain illness by circulating a perfusate (perfusing fluid) in a body cavity of an object including human beings, where the circulated perfusate has been heated to a temperature that is higher than the normal body temperature of the object. One particular hyperthermia treatment is the chemohyperthermia treatment that is a fusion of chemotherapy and hyperthermia treatment. For chemohyperthermia treatment, the perfusing fluid in the body cavity is heated up to 45° C. to increase the susceptibility of cancer cells in the body cavity to the chemotherapeutic agents. Chemohyperthermia treatment has been used as an adjunct therapy for cancer patients because it increases the survival rate of patients significantly and improves the quality of patients' life.

It has been established that chemohyperthermia treatment is very effective for the treatment of peritoneal cancer. One method of applying chemohyperthermia treatment is by perfusion of heated liquids (perfusate) into a body cavity of an object, which is known as intracavitary chemohyperthermia. An example of intracavitary chemohyperthermia is intraperitoneal chemohyperthermia (IPCH) treatment that circulates perfusate through the peritoneum. One known IPCH system is the ThermoChem HT-1000 from ViaCirq Inc. (US). The ThermoChem system is used to provide an adjunctive treatment that continuously circulates preheated perfusion fluid throughout the peritoneum, thereby increasing the temperature of the peritoneal cavity up to 45° C.

During chemohyperthermia treatment, it is critical to maintain the temperature of perfusate being introduced into a body cavity of an object. In order to do so, the ThermoChem system uses a water bath heating system to heat the perfusion fluid. The water bath heating system comprises a heat exchanger that uses convective heat transfer between a discrete circulated water bath and the perfusate to indirectly heat the perfusion fluid that is circulated into the patient's body. Although the ThermoChem system provides consistent heating of the perfusion fluid, it has certain drawbacks. For example, the water bath heating system requires additional components such as a water tank and water pump control modules. These additional components are usually housed in a separate compartment in order to prevent spillage into the main control system. As a result, the additional components and compartment increase the weight and profile of the ThermoChem system significantly. For example the ThermoChem system has a weight of 155 kg, height of 1.7 m, and width of 0.85 m.

Another treatment that is gaining prominence is the perfusion of the bladder in order to destroy the carcinoma in the bladder. In bladder cancer treatment, the rate of tumor recurrence and the disease progression has been high among intermediate-to-high risk patients, indicating that chemotherapy alone is not sufficient. Therefore chemohyperthermia treatment has gained popularity recently. The treatment is similar to the irrigation of the peritoneal; however the bladder treatment differs in that the volume of perfusate is limited to the bladder itself. Currently, the most common treatment is to fill the bladder with chemotherapeutic fluid and then drain the bladder at the end. A recent development is directed towards the heating of the bladder cavity. In this treatment, a radiofrequency probe, together with the inflow/outflow catheter, is inserted into the bladder. Thus, the radiofrequency radiation emitted heats up the fluid and bladder. Meanwhile the system flushes the bladder with the perfusate. This requires very advance control and manipulation of radiofrequency radiation to ensure uniform and stable heating. Moreover, a slight miscalculation or nonperforming temperature sensor could result in harm.

### SUMMARY OF THE INVENTION

In a first aspect, the invention provides a system for chemohyperthermia treatment comprising a reservoir for storing perfusion fluid; a heating/cooling assembly in communication with the reservoir so that the perfusion fluid can be transferred from the reservoir to the heating/cooling assembly, wherein the heating/cooling assembly comprises: at least one heating unit in heat transfer communication; a channel through which the perfusion fluid passes, the channel having an inlet for in-flowing the fluid and an outlet for out-flowing the fluid; said at least one heating unit operable in a heating mode or cooling mode; such that in the heating mode, the at least one heating unit heats the fluid flowing through the channel, and in the cooling mode, said at least one unit cools the fluid flowing through the channel; a heat conductive member placed intermediate the at least one heating unit and the channel; a pumping means coupled to the heating/cooling system, wherein the pumping means pump the perfusion fluid from the reservoir to the heating/cooling system, thereby allowing the heating/cooling system to change the temperature of the fluid; at least one inflow catheter coupled to the pumping means, wherein the at least one inflow catheter delivers the heated/cooled fluid to an object; and at least one outflow catheter coupled to the reservoir, wherein the at least one outflow catheter drains the fluid from the object to the reservoir.

In a second aspect a system for chemohyperthermia treatment comprising: a reservoir for storing fluid; a heating/cooling system coupled to the reservoir so that the fluid can be transferred from the reservoir to the heating system, wherein the heating/cooling system comprises: a heating/cooling exchange module having a channel within which the fluid can flow; wherein the channel has an inlet for in-flowing the fluid and an outlet for out-flowing the fluid; and a plurality of peltier modules coupled to the heating/cooling module, wherein each of the plurality of peltier modules can operate in a heating mode or cooling mode independently; wherein in the heating mode, the plurality of peltier modules heat up the fluid flowing through the channel, and wherein in the cooling mode, the plurality of peltier modules cool the fluid flowing through the channel; a pumping means coupled to the heating/cooling system, wherein the pumping means pump the perfusion fluid from the reservoir to the heating/cooling system, thereby allowing the heating/cooling system to change the temperature of the fluid; at least one inflow catheter coupled to the pumping means, wherein the at least one inflow catheter delivers the heated/cooled fluid to an object; and at least one outflow catheter coupled to the reservoir, wherein the at least one outflow catheter drains the fluid from the object to the reservoir.

In a third aspect, the invention provides a method of chemohyperthermia treatment comprising the steps of: pumping perfusion fluid from a reservoir to a heating/cooling assembly; activating at least one heating unit in heat transfer communication with a channel through which the perfusion fluid passes, the channel having an inlet for in-flowing the fluid and an outlet for out-flowing the fluid; heating said perfusion fluid whilst in said assembly; pumping said heated fluid from the assembly to a bodily cavity for which the treatment is intended; circulating said heated fluid within said cavity; removing said fluid from the cavity.

For clarity, in describing the various embodiments of the present invention, inflow shall refer to the flow of liquid into the body and outflow will refer to the flow of liquid out of the body.

In one embodiment of the system, the heating/cooling exchange module may comprise a body having a groove, and a conductor enclosing the groove to form the channel.

In another embodiment of the system, the pumping means and bypass/pinch valves may further regulate the flow rate of the fluid in the system. In this embodiment bypass/pinch valves refer to a valve having either a first inlet that can selectively use either of two outlets, or alternatively have two selectively operable inlets for a single outlet.

In another embodiment of the system, the system may further comprise a tubing coupled between the pumping means and the reservoir, and a bypass switch configured to control the fluid flowing into either the at least one inflow catheter or the reservoir.

In another embodiment of the system, the system may further comprise a mini-reservoir coupled between the heating system and the pumping means to dampen the temperature of the heated fluid.

In another embodiment of the system, the reservoir may comprise an air vent for releasing pressure so that the system can be an open or vented system so as to minimize trauma to the tissue through excessive pressure at the outflow. Alternatively, the system may be maintained as a closed pressurized system to better control the inflow and outflow of the perfusate.

In another embodiment of the system, the heating/cooling system may further comprise a heating/cooling plate disposed between the plurality of peltier modules or direct contact heaters and the heating/cooling exchange module to transfer heat from the plurality of peltier modules, or direct contact heaters, to the heating/cooling exchange module or vice versa. In a further embodiment of the system, the heating/cooling system may further comprise a heat sink coupled to the plurality of peltier modules to dissipate heat from the plurality of peltier modules. In another further embodiment of the system, the heating system may further comprise a plurality of box fans coupled to the heat sink, wherein the plurality of box fans facilitates the dissipation of heat by the heat sink.

In another embodiment of the system, the heat exchange system may have a large surface area which enables an efficient heat transfer rate and correspondingly a reduced temperature gradient between the heater and circulating fluid. In a preferred embodiment, this large surface area may be greater than 20 cm2 for the Intraperitoneal Chemohyperthermia (IPCH) and greater than 10 cm2 for the Intravesical chemohyperthermia (IVCH).

In another embodiment, the system may further comprise a first pressure and temperature sensing probe coupled to the at least one inflow catheter for measuring the pressure and temperature of the fluid flowing into the object. In a further embodiment, the system may further comprise a second pressure and temperature sensing probe coupled to the at least one outflow catheter for measuring the pressure and temperature of the perfusion fluid drained from the object. In another further embodiment, the system may further comprise a third pressure and temperature sensing probe coupled to the heating system to measure the temperature of the fluid heated by the heating system. In yet another further embodiment, the system may further comprise at least one level sensor coupled to the reservoir to detect the level of perfusion fluid in the reservoir, thereby preventing the perfusion fluid from over filling the reservoir or prevent the premature emptying of the perfusion fluid from the selected media. In yet another further embodiment, the system may comprise a series of switch/pinch valves attached to the inflow/outflow catheter which control the flow of the perfusate to and from the body/reservoir, and thereby minimise the risk of overfilling of the reservoir/body.

In another embodiment, the system may further comprise a computer system coupled to the level sensor and the pressure and temperature sensing probes bypass switch/pinch valves wherein the computer system may be programmed to monitor the perfusion fluid level detected by the level sensor, and wherein the computer system can be programmed to monitor and maintain the parameters detected by the pressure and temperature sensing probes. In a further embodiment, the computer system may comprise an interactive display means that enables a user to observe and adjust the system parameters according to different treatment requirements. The interactive display highlights safety warnings and allows user to activate emergency actions, if these emergency actions are not already initiated by the system.

In another embodiment, the pumping means may comprise a plurality of roller, or peristaltic, pumps.

In another embodiment, the system may further comprise self-contained fluid disposable drainage bag for collection of the fluid.

In another embodiment of the system, the chemohyperthermia treatment may be an intracavitary one.

The chemohyperthermia treatment system of the present invention has many advantages. For example, the direct heating and monitoring system may be readily controlled and provide a reliable form of heating of the perfusion fluid. Furthermore, the direct heating system may reduce the number of components of the system. This may have the advantage of reducing the weight and profile platform for the system. Other advantages of this invention will be apparent with reference to the detailed description.

In a further embodiment, said reservoir may be heated reservoir so as to maintain the fluid at, or near, a user defined temperature. This may be achieved by external heaters applied to said reservoir. Alternatively, the reservoir may include a heating element within walls of the reservoir, or within the fluid itself. The perfusion fluid enters the temperature modulator, to receive further heating, by a secondary heating unit. This secondary heating unit may act as temperature modulation, and may provide greater control and/or sensitivity to the fluid temperature, prior to entering the cavity. The power to the components of the secondary heating unit may be regulated by the controller. In this case it may rely on the input of the temperature probe which may measure, to a high level of sensitivity, the temperature of fluid entering the cavity. The fluid is then pumped into the cavity via the catheter which ensures an even dispersal of heated fluid. At the base of the cavity is the outflow catheter which allows the fluid to escape the cavity. The fluid is finally returned to the reservoir.

Temperature probes could be inserted into the cavity to measure the environment and to measure the efficacy of the hyperthermia delivery.

There may be a single line of tubing entering the tract leading to the cavity. Enclosed in this jacket may be the inflow catheter, outflow catheter and temperature probes. Once deployed, the inflow catheters may be situated above and/or near the entrance of the cavity. The outflow may be at the base of the cavity. The temperature probe may be near the wall of the bladder to accurately gauge the temperature of the bladder.

The inflow catheter may have a spray nozzle, or alternatively a rotating sprinkle design that sends the heated perfusion down the side of the cavity. The outflow catheters may be designed to cater for negative pressure, or gravity drainage. In another embodiment, the catheters may enable continuous perfusion in and out of the bladder. In a further embodiment, said continuous perfusion may be at a very low flow rate, so as to require less perfusion fluid in circulation, and consequently to minimize risk of bladder distension.

The IVCH may have two configurations-large volume and small volume, based on the required reservoir volume. In an embodiment including a high volume system, said system may be gravity fed and an open system. In an embodiment including a small volume system, said system may be a closed system, with an option of the reservoir-depending on how small the volume.

### BRIEF DESCRIPTION OF THE DRAWINGS

It will be convenient to further describe the present invention with respect to the accompanying drawings that illustrate possible arrangements of the invention. Other arrangements of the invention are possible, and consequently the particularity of the accompanying drawings is not to be understood as superseding the generality of the preceding description of the invention.

FIG. 1 is a block functional diagram of an intraperitoneal chemohyperthermia (IPCH) system in accordance with one embodiment of the present invention.

FIG. 2 is an exploded view of the direct heating/cooling system in accordance with one embodiment of the present invention.

FIG. 3 is an assembled view of the direct heating/cooling system in FIG. 2.

FIG. 4 is a cross-sectional view of the direct heating/cooling system looking from the line A-A.

FIG. 5 is a block functional diagram of an intravesical chemohyperthermia (IVCH) system in accordance with a further embodiment of the present invention.

FIGS. 6A and 6B are, respectively, an isometric and elevation view of the heating/cooling exchange module according to a further embodiment of the present invention;

FIG. 7 is an elevation view of a probe of the IVCH system according to a further embodiment of the present invention.

FIG. 8 is a cross sectional view of a patient's bladder having the probe of FIG. 7 inserted therein according to a further embodiment of the present invention.

FIGS. 9A and 9B are respectively plan and elevation views of the channel according to a further embodiment of the present invention.

FIGS. 10A and 10B are respectively plan and elevation views of the channel according to a further embodiment of the present invention.

### DETAILED DESCRIPTION OF THE INVENTION

The present invention may be understood more readily by reference to the following detailed description of certain embodiments of the invention.

The present invention provides a chemohyperthermia system that provides stable and consistent heating of the perfusion fluid. While the chemohyperthermia system will be illustrated by the exemplary intraperitoneal chemohyperthermia (IPCH) an intravesical chemohyperthermia (IVCH) treatments in the description hereinafter, it is contemplated that the system may be used for other forms of treatment procedures for instance within the, lungs or limb that require a perfusion fluid/body fluid to be heated up in a controlled manner. Whilst the apparatus for IPCH and IVCH may be similar, the two systems will vary based on application given the different illnesses being treated. For instance, for the IPCH treatment, a flow rate in range 700 ml to 1000 ml per minute is typical using a continuous flow of perfusion fluid into the peritoneal cavity. Given the size of the cavity and also the need for a continuous flow IPCH treatment may incorporate two inflow and two outflow portions which are gravity drained. Being a larger volume, a typical treatment period may be around two hours and conducted under strict supervision such as within an operating theatre.

Alternatively, the IVCH system involves flow rates in the range 200 ml to 500 ml per minute which may involve an intermittent flow of perfusion fluid although continuous flow may also be used. Because of the smaller volume of the bladder cavity, a single inflow and outflow is preferred.

In the IVCH treatment, in addition to gravity drain system, a pressurized system may also be incorporated so as to better control the rates of inflow and outflow and particularly since treatment period should be no more than one hour in light of patient discomfort is also applicable though monitoring of the pressure clearly is more important than IPCH treatment despite an IVCH treatment typically being conducted in an outpatient clinic environment.

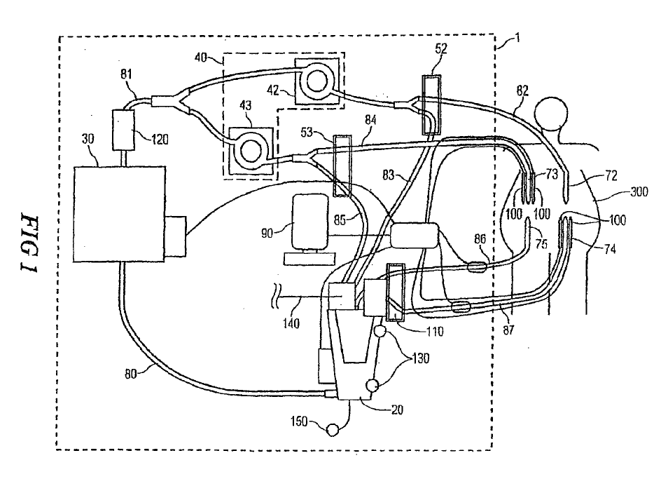

Referring to FIG. 1, there is provided a block functional diagram of an intraperitoneal chemohyperthermia (IPCH) system in accordance with one embodiment of the present invention. The IPCH system 1 comprises a reservoir 20, a direct heating/cooling system 30, a pump system 40, a plurality of bypass switches 52, 53, a plurality of temperature and pressure probes 100, and a plurality of inflow catheters 72, 73 and outflow catheters 74, 75.

The reservoir 20 functions as storage for perfusion fluid. The perfusion fluid that can be used in the present application is not limited any particular therapeutic reagents or composition. For example, the perfusion fluid may be standard peritoneal dialysis solution; and the choice of chemotherapeutic reagents can be determined by the physicians involved in performing the IPCH procedure. In addition, the reservoir 20 may comprise a filtration system for controlling the quality of the perfusion fluid flowing out from the reservoir 20. Furthermore, the reservoir 20 may have an air vent (not shown) for releasing pressure so that the IPCH system 1 can be an “open” or vented system. The reservoir 20 can be of any suitable configurations and dimensions.

The direct heating/cooling system 30 is coupled to the reservoir 20 via a tubing 80, wherein the tubing 80 serves as a channel for transferring perfusion fluid from the reservoir to the heating system 30. The direct heating/cooling system 30 is configured to ensure consistent regulation of temperature and rapid response to any necessary adjustments by using a solid-liquid heating/cooling method.

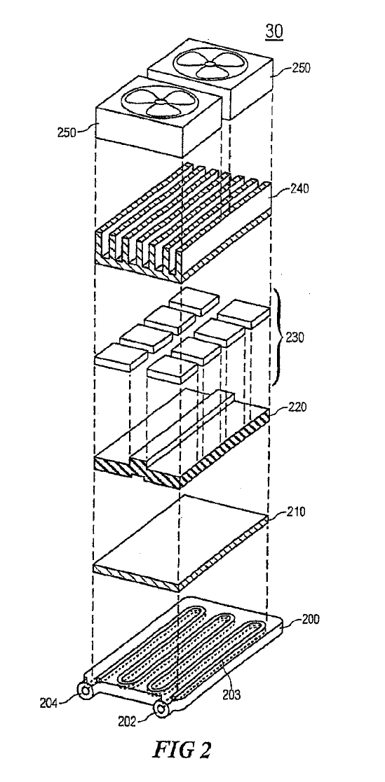

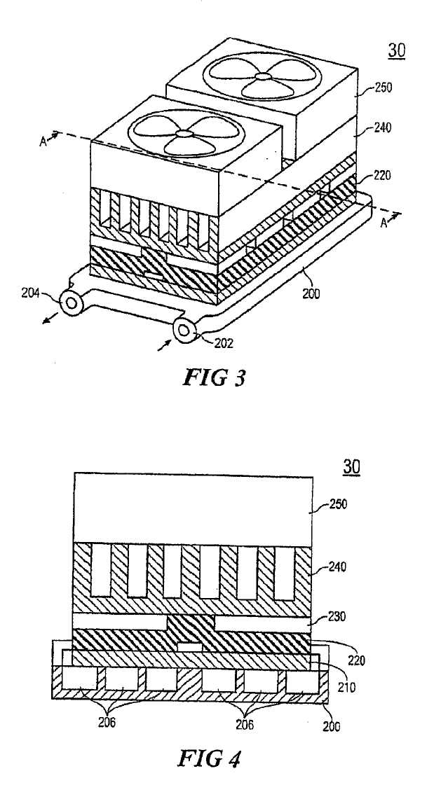

Referring to FIGS. 2-4, there are provided different views (exploded, assembled, or cross-sectional) of the direct heating/cooling system 30 in accordance with one embodiment of the present invention. As shown in FIG. 2, the direct heating/cooling system 30 comprises a heat exchanger 200, a conductor 210, a heating/cooling plate 220, a plurality of peltier modules 230, a heat sink 240, and a plurality of box fans 250.

In this embodiment, the heat exchanger 200 comprises an inlet 202, a groove 203, and an outlet 204. The heat exchanger 200 can be made from any suitable materials including plastics or the like, subject to the heat resistance required of the material. The conductor 210 is coupled to the heat exchanger 200 to enclose the groove 203, thereby forming a fluid channel 206 as shown in FIG. 4. In operation, the inlet 202 receives perfusion fluid from the reservoir 20, wherein the perfusion fluid then flows through the channel 206 and exits from the outlet 204. The plurality of peltier modules and direct heating strips 230 serve as the heating/cooling source for the direct heating/cooling system 30 and are coupled to the conductor 210 via the heating/cooling plate 220. In a heating mode, the plurality of peltier modules 230 heat up the heating/cooling plate 220. Then, the conductor 210 transfers the heat from the heating/cooling plate 220 to the perfusion fluid flowing in the channel 206, thereby heating up the perfusion fluid. The conductor 210 is preferably made from good heat conductivity materials such as aluminum. In a cooling mode, the plurality of peltier modules 230 cool down the heating/cooling plate 220, which in turn cools the conductor 210. As a result, the conductor 210 cools the perfusion fluid flowing through the channel 206. Whilst the arrangement of the heat exchange module 170 may have common features whether arranged for IPCH or IVCH treatment, there will, nevertheless, be modifications varying features to make the heat exchange module 170 more applicable for one treatment over the other. A feature of the channels 206, in this embodiment, is to provide a meandering path through the heat exchange module 170 so as to provide sufficient surface area to impart heat to the perfusion fluid. Given the higher volume of fluid passing through the channels for an IPCH treatment which may be in the range 700 ml to 1000 ml per minute, a higher surface area is adopted such that the channels may have a total surface area of the order of 20 sq cm. Alternatively, for the IVCH treatment having a lower flow rate such as in the range 200 ml to 500 ml per minute, a lower surface area is typically provided such as about 10 sq cm. An alternative form of the channels is to provide a low aspect ratio channel, which is one having a broad base and low height, thus providing a larger surface area in the plane from which the heat is imparted.

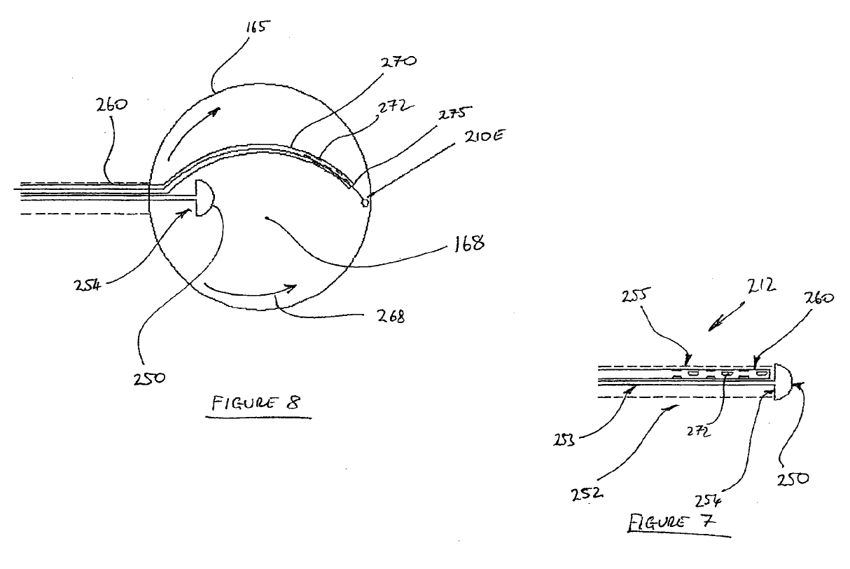

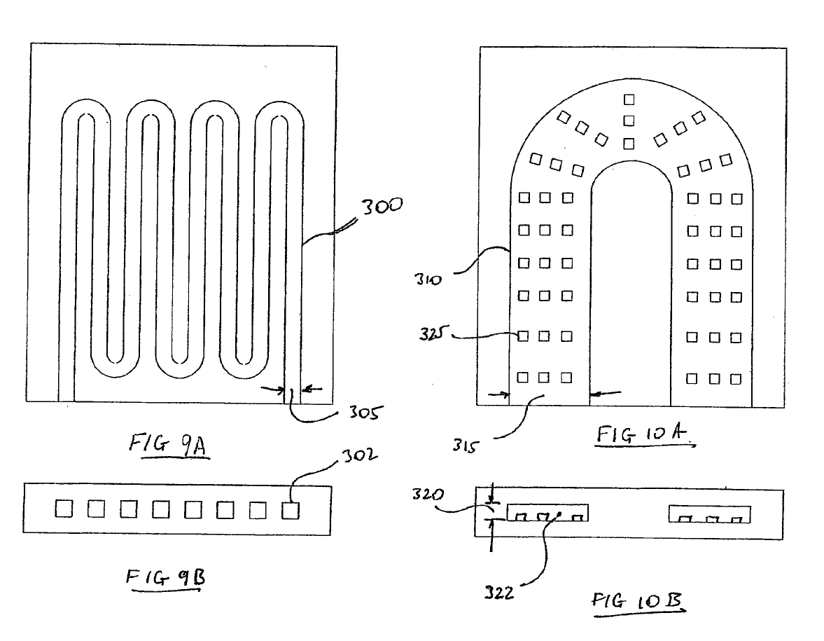

FIGS. 9A,B and 10A,B show representations of the different embodiments of the channel. In particular, FIGS. 9A and 9B provide a schematic view of a “meandering” channel 300 whereby the required surface area is provided by having a long path with a cross sectional shape 302 having height and width of approximately the same dimension. FIGS. 10A and 10B show the alternative view, whereby the cross sectional shape 322 has a low aspect ratio, that is, the width 315 is much greater than the height 320. In a further embodiment, further surface area is provided by “profiling” the base of the channel 310 through adding ridges or baffles 325 through which the fluid must pass. Thus, greater temperature uniformity is achieved due to the increase turbulence and mixing of fluid caused by the baffles on the base of the channel.

In one embodiment, each of the plurality of peltier modules 230 has a first surface and second surface. While the first surfaces of the plurality of peltier modules 230 are coupled to the heating/cooling plate 220, the second surfaces of the plurality of peltier modules 230 are coupled to the heat sink 240. When in use, a voltage can be applied to the plurality of peltier modules 230 to achieve a temperature difference between the first surface and second surface of each peltier module 230. For example, the first surface can be hot and the second surface can be cold to achieve the heating mode. In the heating mode, the hot first surface of each peltier module 230 heats up the heating/cooling plate 220. As a result, the perfusion fluid flowing in the channel 206 is heated up as discussed above. In the cooling mode, the polarity of the voltage applied to the plurality of peltier modules 230 are simply reversed; thus the first surface of each peltier module 230 is cold and the second surface is hot. The cold first surface of each peltier module 230 cools down the heating/cooling plate 220. As a result, the perfusion fluid flowing in the channel 206 is cooled down. In addition, the heat sink 240 dissipates the heat away from the hot second surface of each peltier module 230. The plurality of box fans 250 coupled to the heat sink 240 facilitates the dissipation of heat from the second surface of each peltier module 230.

The advantages of the direct heating/cooling system using the peltier module 230 are evident. For example, the direct heating/cooling system is a solid-state device with no moving parts, resulting in extreme reliability and little or no maintenance requirement. The peltier module 230 provides the desired, stable and consistent heating/cooling of the perfusion fluid by means of both hardware and software control. Furthermore, the use of the peltier module 230 reduces the total amount of components for the IPCH system 1 by eradicating the need for a bulky water tank and heat exchanger. Thus, the IPCH system 1 with its solid to liquid direct heating/cooling system 30 has a smaller weight and smaller profile platform as compared to the conventional systems using water tanks and heat exchangers.

Referring back to FIG. 1, the direct heating/cooling system 30 is coupled to the pump system 40 via the tubing 81. The pump system 40 controls the flow rate of the perfusion fluid for effective perfusion and dispersion. During operation, the pump system 40 pumps the perfusion fluid from the reservoir 20 to the direct heating/cooling system 30, wherein the perfusion fluid enters the inlet 202 of the heat exchanger 200 and flows through the channel 206. As the perfusion fluid is flowing through the channel 206, it is being heated or cooled by the plurality of peltier modules 230. Thereafter, the heated/cooled perfusion fluid exits the heat exchanger 200 from the outlet 204 and is transferred to the pump system 40. In a preferred embodiment, the pump system 40 comprises a first roller pump 42 and a second roller pump 43 that are configured to transfer the heated/cooled perfusion fluid from the direct heating/cooling system 30 into the peritoneal cavity 300. The two roller pumps 42, 43 provide a more effective and efficient perfusion fluid distribution into the patient's peritoneal cavity 300.

The first roller pump 42 is coupled to a first bypass switch 52, wherein the first bypass switch 52 is coupled to a first inflow catheter 72 via inflow tubing 82. Furthermore, the first bypass switch 52 is coupled to the reservoir 20 via inflow bypass tubing 83. The second roller pump 43 is coupled to a second bypass switch 53, wherein the second bypass switch 53 is coupled to a second inflow catheter 73 via tubing 84. Furthermore, the second bypass switch 53 is coupled to the reservoir 20 via second bypass tubing 85. The first and second inflow catheters (72, 73) deliver the heated/cooled perfusion fluid to the peritoneal cavity 300. Thereafter, the perfusion fluid is drained from the peritoneal cavity via outflow catheters 74, 75. The outflow catheters 74, 75 are coupled to the reservoir 20 via outflow tubings 86, 87, wherein the tubings 86, 87 transfer the perfusion fluid from the peritoneal cavity 300, referred herein as the peritoneal perfusate, to the reservoir 20. An automated gross pinch valve 110 can be coupled to the tubings 86, 87 to control the outflow of the peritoneal perfusate. The level of the perfusate in the reservoir 20 can be monitored by a high and low level sensor 130. Furthermore, the reservoir 20 acts as a gross filter to the peritoneal perfusate before transferring the filtered perfusion fluid to the direct heating/cooling system 30. In addition, perfusion fluid can be added to the reservoir 20 if necessary via an attachment tubing 140 connected to the inlet of the reservoir 20.

The first and second bypass switches (52, 53) allow the internal circulation of the perfusion fluid within the IPCH system 1. Internal circulation of the perfusion fluid allows it to be pre-heated to a desired temperature before being re-directed towards the inflow catheters (72,73) for circulation within the patient's peritoneal cavity 300. The operations of the first and second bypass switches (52, 53) are controlled by a computer system 90. When the computer system 90 detects a bypass event such as breach of safety levels, temperature, pressure or an abnormally high temperature reading from the direct heating/cooling system 30, the system 90 activates the first and second bypass switches (52, 53) to open the tubings 83, 85 and close the tubings 82, 84, 86, 87. In this case, the perfusion fluid from the pumping system 40 is directed to the reservoir 20, thereby preventing the high temperature perfusion fluid from entering the patient's peritoneal cavity 300.

Still referring to FIG. 1, a secondary safety device (mini reservoir) 120 is disposed immediately after the direct heating/cooling system 30 to ensure that any high temperature perfusion fluid is mixed adequately to reduce the temperature before it is transferred to the inflow catheters 72,73.

The drainage of the peritoneal perfusate from the peritoneal cavity 300 into the reservoir 20 may be achieved by the concept of gravitational siphoning or open system. Conventional IPCH systems uses closed system, wherein the closed system is not vented to equalized at atmospheric pressure from the patients' peritoneal cavity to the roller pump due to a lack of a vented reservoir. The advantage of an open system is that it helps to prevent negative pressure or sucking of organs and/or tissues located near the outflow catheters. As a result, tissue trauma due to the outflow catheters is reduced during the treatment process. In the present invention, the peritoneal perfusate is drained passively by the gravitational pull to the vented reservoir 20. As a result, the bare minimum negative suction is created by the siphoning effects of gravity and not by the uncontrolled actively created negative suction from the roller pumps. Gross pinch valves are added to the outflow catheters to prevent the drainage of the peritoneal perfusate from the peritoneal cavity into the reservoir in the event of internal circulation, overfilling of the reservoir is detected by the level sensors or when the pumps are switch off.

Still referring to FIG. 1, the IPCH system 1 may further comprise a self-contained fluid disposable drainage bag system 150 that is used to collect fluid media in a safe manner for the operator. This minimizes the operator's risk of coming into contact with the contaminated chemical/biological fluid at the end of each treatment procedure. The inflow catheters (72, 73), outflow catheters (74, 75), and related perfusion apparels such as the tubings (80-87) are designed to be of sterile single use sets and can be made to be disposable at the end of each treatment procedure.

A plurality of pressure sensitive and temperature sensitive probes 100 are disposed at the inflow catheters (72, 73) and outflow catheters (74, 75) to monitor the operating pressure and temperatures so as to ensure patient safety. The plurality of probes 100 can be controlled by the computer system 90. Furthermore, the probe 100 can be disposed at the tubing 81 between the direct heating/cooling system 30 and the pumping system 40. The probe 100 is provided at the tubing 81 to ensure that the heated perfusion fluid is within safety limits. Another probe 100 can be deployed after the roller pumps (42, 43) to detect insidious or acute build up of pressure. Upon detection of this build up, the system software 90 will shut the roller pumps (42, 43) in order to prevent the bursting of tubings and thus keeping the integrity of the tubings for the continuation of the procedure upon physical rectification of the pressure build up by either the attending surgeon or perfusionist.

Temperature measurements probes 100 can be any available temperature measurement devices/technologies. In some embodiments, the temperature measurements probes 100 utilize Resistance Temperature Detector (RTD) technology or Thermocouples Technology as a feedback temperature control. In other embodiments, the temperature probes can be inside the tube, in the cavity or around the tubing but external to the cavity.

The computer system 90 is coupled to the plurality of probes 100 for monitoring the temperature and pressure. Furthermore, the computer system 90 can be coupled to the high and low level sensors 130 to monitor the level of perfusion fluid in the reservoir 20. Most importantly, the controller 90 is coupled to the direct heating/cooling system 30 for controlling the heating of the perfusion fluid. The controller 90 can be coupled to an interactive display interface (not shown) such as a touch screen monitor that enables the operator to define and monitor the system parameters accordingly to its embedded software control.

The system 1 may be operated by a perfusionist or other professionals who are trained in perfusion management as the characteristic of perfusion management techniques in open heart surgery is similar in requirements to that of an IPCH procedure. Typically, a perfusionist uses the heart lung bypass machine that is ergonomically designed. The heart lung machine enables the perfusionist when seated on a stool to have a global view of the main circulatory components such as the pump, reservoir (which oxygenates the blood) and inflow/outflow tubes. More importantly, the low profile of the machine enables the perfusionist to have an extended and unobstructed view of the operating procedure/setting, patient monitor and concurrent to manipulating the heart lung machinery. With lesser components, the IPCH system 1 tries incorporates the characteristic profile of that of the standard heart/lung setup in terms of low height profile, ergonomic positioning of components, device mobility and user-interface. IPCH system 1 is low in height and weighs one third less than the other devices in the market.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the spirit and scope of the present invention. Accordingly, the scope of the present invention is described by the appended claims and is supported by the foregoing description.

In a further embodiment of the present invention, the apparatus may be configured so as to provide intravesical chemohyperthermia treatment (IVCH).

FIG. 5 shows such an embodiment whereby the invention has been configured so as to provide perfusion fluid to the bladder. In particular FIG. 5 shows a schematic of the system 160 whereby a reservoir 175 of the perfusion fluid is in communication with a pump 180, such as a peristaltic pump. The pump 180 provides the fluid to a heat exchange module 170 which heats the fluid as it moves to the bladder 165. A probe 212 is inserted into the bladder for the inflow of perfusion fluid and subsequently removed so as to dispose of the used fluid, re-circulate 200 to the reservoir 175 or maintain a pressurized system by feeding 205 the fluid back to the pump 180. An important feature of this and other embodiments of the present invention is to maintain, measure and control temperature of the fluid throughout the active or upstream side of the system. To this end temperature sensors may be placed around the system at strategic locations.

FIG. 5 further shows the placement of said temperature sensors 210A to E placed at various points around the system. For instance, one sensor 210A may be placed in the reservoir 175 to monitor the temperature of the perfusion fluid supply. It follows that control of the system will require information on the perfusion fluid supply temperature provided by the temperature sensor 210A so as to determine the required temperature and residence time of the fluid within the heat exchange module 170.

In one embodiment, to minimize the temperature gradient of the fluid through the heat exchanger 170 both in terms of applied heat and residence time, it may be advantageous to apply heat to the reservoir 175. In so doing, the fluid leaving the reservoir and delivered to the heat exchange module 170 is already of a temperature approaching the required temperature. Thus, the heat exchange module 170, instead of providing the full measure of heat required by the fluid, it may merely act as a final control, and so for increase the sensitivity of control of the fluid temperature prior to inflow into the bladder 165.

The system further provides for an overflow or diversion 190 located after the heat exchange module 170 and feeding back to the reservoir. The diversion 190 is intended to limit or prevent the build up of heat in the heat exchanger and as well as to maintain the temperature of the fluid in the reservoir by providing heated fluid directly from the heat exchange module 170 to the reservoir 175. In a further embodiment, the diversion may also operate such that fluid being pumped to the heat exchange module 170 does not exceed the flow by which the heat exchange module 170 can impart heat under a controlled manner. Accordingly, should the sensitivity of the pump 180 not be sufficient to match the sensitivity of the heat exchange module 170, the diversion 190 can remove excess fluid back to the reservoir 175 without “flooding” the heat exchange module 170.

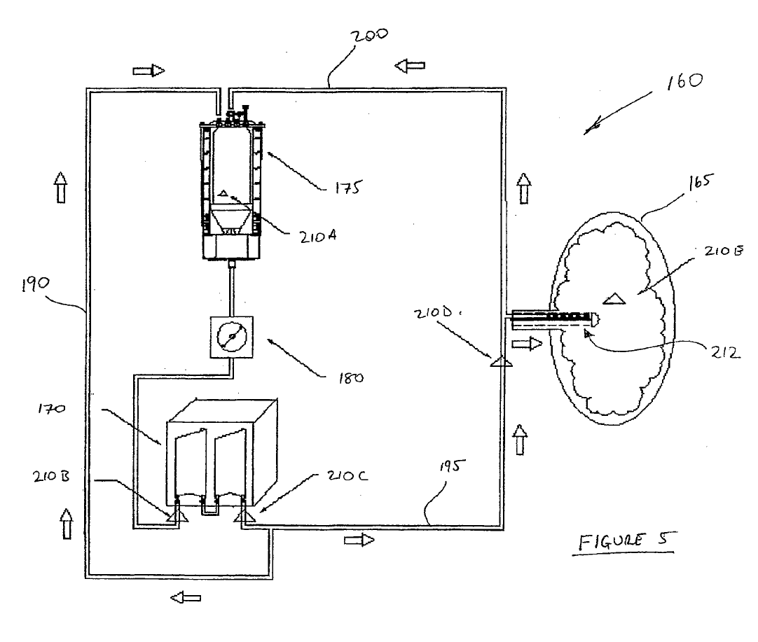

FIGS. 6A and 6B show an alternative arrangement of the heat exchange module 170 as compared to that shown in FIG. 4. In this arrangement, instead of a heater 220 being in heat communication with a conductor 210, a dual heating arrangement 222A, B is provided, separated by a conductor plate 223 so as to increase the heating potential of the assembly.

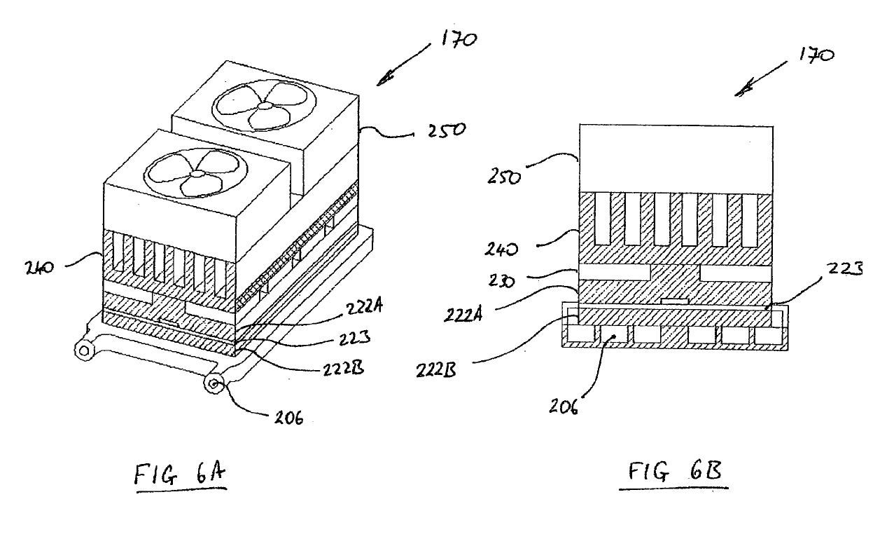

FIG. 7 shows a probe used in accordance with an embodiment of the present invention to be inserted into the bladder. The probe 212 comprises a projection 252 having a leading head 250 which is used to insert into the bladder 165. The head 250 is engaged with a sleeve 255 and within said sleeve is located a sensor tube 260 carrying a temperature sensor for deployment within the bladder. The probe 212 includes a hollow shaft 253 through which the perfusion fluid flows to exit from an orifice 254 positioned at a face opposed to a leading portion of the head 250. The leading head 250 acts as the inflow catheter. The hemispherical shape of the leading head 250 is intended to provide a greater area for the orifice 254 so as to more evenly distribute the heated perfusion fluid across the walls of the bladder. The sensor tube 260 on deployment within the bladder then acts as an outflow catheter for the perfusion fluid through the array of holes 272 placed in the tube.

FIG. 8 shows the deployment of the probe 212 whereby the head 250 has projected into the bladder 165 so as to permit perfusion fluid to flow from the orifice 254 and circulate around the bladder 165. The sensor tube 260 has been further projected so as to extend away from the probe 212 such that the leading end 275 of the tube 260 is located at a distal end of the bladder 165 within the cavity 168. The head 250 of the probe 212, however, remains adjacent to the entry to the bladder 165 such that the perfusion fluid flows from one end of the bladder and the sensor tube located at the far end. Further, the sensor tube then projects a temperature sensor 210E from the opening 275 of the tube 260 with the arrangement such that the temperature of the perfusion fluid is measured after exiting from the orifice 254 and having flowed around the bladder cavity 168 before measuring said temperature. Thus, temperature readings from the sensor 210E are not distorted from measuring the temperature of the fluid still within the tube but instead of fluid flowing within the cavity 168.

Severe bladder distension can cause severe complication. The greatest concern would be spillage of the chemotherapy into the kidney. Hence the need to control the volume of fluid in the bladder to the minimum. Furthermore, the patient does produce urine during the procedure, which adds to the volume.

The foreseeable difficulty is deciding which configuration to adopt. The critical criteria are the ability to distribute heat and chemotherapy uniformly around the bladder.

The present invention does not require a heating element present in the cavity. The challenge is to ensure that fluid entering is at 42 degrees Celsius (or user defined) and that hyperthermia is indeed induced. It follows that a higher flow rate must be achieved to increase the efficacy of the treatment. One advantage is that the period of treatment could be extended since no active (energy emitting) is present.

Whilst both falling within the broader invention, the embodiments encompassing the IVCH and IPCH systems differ from each other in the nature of at least some of their functional aspects, for instance the disposable circuit differs markedly. The bladder is small compared to other cavities with the entry being even smaller, thus the inflow and outflow catheters are much smaller. The inflow has to ensure uniform distribution of the heated fluid without a massive build-up of fluid that could cause a distension of the bladder resulting in the patient feeling severe discomfort. Similarly, the outflow has to be efficient to prevent a scenario of severe bladder distension.

What is claimed is:

1. A system for chemohyperthermia treatment comprising:
a reservoir for storing perfusion fluid;
a heating/cooling assembly in communication with the reservoir so that the perfusion fluid can be transferred from the reservoir to the heating/cooling assembly, wherein the heating/cooling assembly comprises:
at least one heating unit in heat transfer communication a channel through which the perfusion fluid passes, the channel having an inlet for in-flowing the fluid and an outlet for out-flowing the fluid;
said at least one heating unit operable in a heating mode or cooling mode; such that in the heating mode, the at least one heating unit heats the fluid flowing through the channel, and in the cooling mode, said at least one unit cools the fluid flowing through the channel;
a heat conductive member placed intermediate the at least one heating unit and the channel;
a pumping means coupled to the heating/cooling system, wherein the pumping means pump the perfusion fluid from the reservoir to the heating/cooling system, thereby allowing the heating/cooling system to change the temperature of the fluid;
at least one inflow catheter coupled to the pumping means, wherein the at least one inflow catheter delivers the heated/cooled fluid to an object; and
at least one outflow catheter coupled to the reservoir, wherein the at least one outflow catheter drains the fluid from the object to the reservoir.

2. The system of claim 1, wherein said channel includes any one of a tube, a groove formed in said heat conductive member and a low aspect ratio channel.

3. The system of claim 1, further including a heat source applied to said reservoir for heating the stored perfusion fluid while in the reservoir.

4. The system of claim 1, wherein the pump is a peristaltic pump.

5. The system of claim 1, wherein the at least one heating unit includes a peltier module.

6. The system of claim 1, wherein the pumping means further regulates the flow rate of the fluid in the system.

7. The system of claim 1, further comprising a tubing coupled between the pumping means and the reservoir, and a bypass switch configured to control the fluid flowing into either the at least one inflow catheter or the reservoir.

8. The system of claim 1, further comprising a mini-reservoir coupled between the heating assembly and the pumping means to dampen the temperature of the heated fluid.

9. The system of claim 1, wherein the assembly further comprises a heat sink coupled to the at least one heating unit to dissipate heat from said at least one unit.

10. The system of claim 1, further comprising a first pressure and temperature sensing probe coupled to the at least one inflow catheter for measuring the pressure and temperature of the fluid flowing into the object.

11. The system of claim 10, further comprising a second pressure and temperature sensing probe coupled to the at least one outflow catheter for measuring the pressure and temperature of the perfusion fluid drained from the object.

12. The system of claim 10, further comprising a third pressure and temperature sensing probe coupled to the heating system to measure the temperature of the fluid heated by the heating system.

13. The system of claim 10, further comprising a level sensor coupled to the reservoir to detect the level of perfusion fluid in the reservoir, thereby preventing the perfusion fluid from over filling the reservoir or prevent the premature emptying of the perfusion fluid from the selected media.

14. The system of claim 13, further comprising a computer system coupled to the level sensor and the first, second and third pressure and temperature sensing probes, wherein the computer system can be programmed to monitor the perfusion fluid level detected by the level sensor, and wherein the computer system can be programmed to monitor the temperature and pressure detected by the first, second and third pressure and temperature sensing probes.

15. A system for chemohyperthermia treatment comprising:
a reservoir for storing fluid;
a heating/cooling system coupled to the reservoir so that the fluid can be transferred from the reservoir to the heating system, wherein the heating/cooling system comprises:
a heating/cooling exchange module having a channel within which the fluid can flow; wherein the channel has an inlet for in-flowing the fluid and an outlet for out-flowing the fluid; and
a plurality of peltier modules coupled to the heating/cooling module, wherein each of the plurality of peltier modules can operate in a heating mode or cooling mode independently; wherein in the heating mode, the plurality of peltier modules heat up the fluid flowing through the channel, and wherein in the cooling mode, the plurality of peltier modules cool the fluid flowing through the channel;
a pumping means coupled to the heating/cooling system, wherein the pumping means pump the perfusion fluid from the reservoir to the heating/cooling system, thereby allowing the heating/cooling system to change the temperature of the fluid;
at least one inflow catheter coupled to the pumping means, wherein the at least one inflow catheter delivers the heated/cooled fluid to an object; and
at least one outflow catheter coupled to the reservoir, wherein the at least one outflow catheter drains the fluid from the object to the reservoir.

16. The system of claim 15, wherein said channel includes any one of a tube, a groove formed in said heat conductive member and a low aspect ratio channel.

17. The system of claim 1, wherein the chemohyperthermia treatment is Intraperitoneal chemohyperthermia.

18. The system of claim 1, wherein the chemohyperthermia treatment is Intravesical chemohyperthermia.

19. The system of claim 17, wherein the surface area of the channel is at least 20 cm².

20. The system of claim 17, wherein the flow rate is in the range 500 to 1500 ml per minute.

21. The system of claim 18, wherein the surface area of the channel is at least 10 cm².

22. The system of claim 18, wherein the flow rate is in the range 200 to 500 ml per minute.

23. The system of claim 17, wherein the channel is of a length in the range 50 to 100 cm.

24. The system of claim 18, wherein the channel is of a length in the range 100 to 200 cm.

25. A method of chemohyperthermia treatment comprising the steps of:
pumping perfusion fluid from a reservoir to a heating/cooling assembly;
activating at least one heating unit in heat transfer communication with a channel through which the perfusion fluid passes, the channel having an inlet for in-flowing the fluid and an outlet for out-flowing the fluid;
heating said perfusion fluid whilst in said assembly;
pumping said heated fluid from the assembly to a bodily cavity for which the treatment is intended;
circulating said heated fluid within said cavity;
removing said fluid from the cavity.
